(19) United States
(12) Patent Application Publication
(10) Pub. No.: US 2018/0305683 A1
(43) Pub. Date: Oct. 25, 2018

(54) MULTIPLEXED TAGMENTATION

(71) Applicant: Agilent Technologies, Inc., Santa Clara, CA (US)

(72) Inventor: Nicholas M Sampas, San Jose, CA (US)

(21) Appl. No.: 15/938,306

(22) Filed: Mar. 28, 2018

Related U.S. Application Data
(60) Provisional application No. 62/487,359, filed on Apr. 19, 2017.

(51) Int. Cl. C12N 15/10; C12Q 1/6869; C12N 9/96; C12N 9/12

(52) U.S. Cl. CPC C12N 15/1065; C12N 9/1241; C12N 9/96; C12Q 1/6869

(57) ABSTRACT

Described herein, among other things, is a method for amplifying a nucleic acid sample. In some embodiments this method may comprise (a) tagmenting the nucleic acid sample with a population of transposase complexes, wherein the population of transposase complexes comprise: i. a transposase and ii. a set of adaptors of the formula X-Y, wherein: region X is a variable sequence that has a complexity of n, wherein n is at least 3, and region Y is a recognition sequence for the transposase, and (b) amplifying the tagged fragments using a set of primers, wherein the set of primers comprises at least n different primers and the n different primers collectively hybridize to all of the different sequences of region X, or a complement thereof.

### CROSS-REFERENCE TO RELATED APPLICATIONS

This application claims benefit of the filing date of and right of priority to U.S. Provisional Application No. 62/487,359, filed Apr. 19, 2017, which is incorporated by reference herein.

### BACKGROUND

Next-generation Sequencing (NGS) technologies have made whole-genome sequencing (WGS) routine, and various target enrichment methods have enabled researchers to focus sequencing power on the most important regions of interest. However, there is still a need for better methods for making NGS sequencing libraries. For example, genomic DNA can be prepared for next-generation sequencing (NGS) by “tagmentation”, where the transposase simultaneously causes staggered double-stranded breaks in the genomic DNA and adds small oligonucleotide tags on the ends of the fragments. However, one problem with this method is that it requires that there be different tags on the two ends of any particular fragment after tagmentation in order to get PCR amplification, since fragments with the same sequences at both ends will not adequately PCR due to suppression PCR effects. However, in many methods, in order to obtain fragments that have different sequences on their ends, two different sequences are be loaded onto the transposase before tagmentation. Since each end gets randomly tagged, there is a 50% chance that both ends of a fragment will have the same sequence added. These fragments, i.e., the fragments that been tagged with the same sequence at both ends, cannot be amplified efficiently and are not sequenced.

### SUMMARY

Described herein, among other things, is a method for amplifying a nucleic acid sample. In some embodiments, the method comprises tagmenting the nucleic acid sample with a population of transposase complexes, wherein the population of transposase complexes comprise a transposase and a set of adaptors of the formula X-Y, where region X is a variable sequence that has a complexity of n, where n is at least 3, and region Y is a double-stranded recognition sequence for the transposase. This step results in the production of a collection of fragments that are tagged with the variable sequence. Next, the method may comprise amplifying the tagged fragments using a set of primers, wherein the set of primers comprises at least n different primers and the n different primers collectively hybridize to all of the different sequences of region X, or a complement thereof, to produce amplification products. Kits and compositions for practicing the method are also provided.

The compositions, methods and kits described herein find particular use in performing copy number analysis on samples of DNA in which the amount of DNA is limited and/or analysis of samples that contain fragments having a low copy number mutation (e.g. a sequence caused by a mutation that is present at low copy number relative to sequences that do not contain the mutation).

Depending on how the method is implemented, the method can improve the efficiency of tagmentation using multiple PCR primers (>2) to amplify the tagmented products and enabling the use of molecular barcoding by tagmentation while eliminating the need for enzymatic whole genome amplification. As will be described in greater detail below, the method can be applied to sample barcoding, molecular barcoding and phasing of adjacent paired-end reads from the same target DNA duplex (for haplotype sequencing). This present approach should be compatible with duplex sequencing, where both the top and bottom DNA strands are tagged with the same molecular barcode.

### BRIEF DESCRIPTION OF THE FIGURES

The skilled artisan will understand that the drawings, described below, are for illustration purposes only. The drawings are not intended to limit the scope of the present teachings in any way.

FIG. 1 schematically illustrates some of the features of an embodiment of the present adaptor.

FIG. 2 schematically illustrates some features of an embodiment of the present method.

FIG. 3 shows how adjacency-barcoded oligonucleotides can be constructed and used for tagmentation.

FIG. 4 shows the representations of eight index sequences in a sequencing run.

### DEFINITIONS

Before describing exemplary embodiments in greater detail, the following definitions are set forth to illustrate and define the meaning and scope of the terms used in the description.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below.

However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, A., Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The term “nucleic acid sample,” as used herein denotes a sample containing nucleic acids. A nucleic acid samples used herein may be complex in that they contain multiple different molecules that contain sequences. Genomic DNA samples from a mammal (e.g., mouse or human) are types of complex samples. Complex samples may have more than 10⁴, 10⁵, 10⁶ or 10⁷ different nucleic acid molecules. Also, a complex sample may comprise only a few molecules, where the molecules collectively have more than 10⁴, 10⁵, 10⁶ or 10⁷ or more nucleotides. A DNA target may originate from any source such as genomic DNA, or an artificial DNA construct. Any sample containing nucleic acid, e.g., genomic DNA made from tissue culture cells or a sample of tissue, may be employed herein.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution and a number of different elements attached to a solid support at random positions (i.e., in no particular order). A mixture is not addressable. To illustrate by example, an array of spatially separated surface-bound polynucleotides, as is commonly known in the art, is not a mixture of surface-bound polynucleotides because the species of surface-bound polynucleotides are spatially distinct and the array is addressable.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylene carbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid”, or “UNA”, is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability, but retain an ability to base pair with naturally occurring C and G residues, respectively. Unstructured nucleic acid is described in US20050233340, which is incorporated by reference herein for disclosure of UNA.

The term “oligonucleotide” as used herein denotes a single-stranded multimer of nucleotide of from about 2 to 200 nucleotides, up to 500 nucleotides in length. Oligonucleotides may be synthetic or may be made enzymatically, and, in some embodiments, are 30 to 150 nucleotides in length. Oligonucleotides may contain ribonucleotide monomers (i.e., may be oligoribonucleotides) or deoxyribonucleotide monomers, or both ribonucleotide monomers and deoxyribonucleotide monomers. An oligonucleotide may be 10 to 20, 11 to 30, 31 to 40, 41 to 50, 51-60, 61 to 70, 71 to 80, 80 to 100, 100 to 150 or 150 to 200 nucleotides in length, for example.

The term “primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers are generally of a length compatible with their use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

Primers are usually single-stranded for maximum efficiency in amplification, but may alternatively be double-stranded. If double-stranded, the primer is usually first treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

The term “hybridization” or “hybridizes” refers to a process in which a nucleic acid strand anneals to and forms a stable duplex, either a homoduplex or a heteroduplex, under normal hybridization conditions with a second complementary nucleic acid strand, and does not form a stable duplex with unrelated nucleic acid molecules under the same normal hybridization conditions. The formation of a duplex is accomplished by annealing two complementary nucleic acid strands in a hybridization reaction. The hybridization reaction can be made to be highly specific by adjustment of the hybridization conditions (often referred to as hybridization stringency) under which the hybridization reaction takes place, such that hybridization between two nucleic acid strands will not form a stable duplex, e.g., a duplex that retains a region of double-strandedness under normal stringency conditions, unless the two nucleic acid strands contain a certain number of nucleotides in specific sequences which are substantially or completely complementary. “Normal hybridization or normal stringency conditions” are readily determined for any given hybridization reaction. See, for example, Ausubel et al., Current Protocols in Molecular Biology, John Wiley & Sons, Inc., New York, or Sambrook et al., Molecular Cloning: A Laboratory Manual, Cold Spring Harbor Laboratory Press. As used herein, the term “hybridizing” or “hybridization” refers to any process by which a strand of nucleic acid binds with a complementary strand through base pairing.

A nucleic acid is considered to be “selectively hybridizable” to a reference nucleic acid sequence if the two sequences specifically hybridize to one another under moderate to high stringency hybridization and wash conditions. Moderate and high stringency hybridization conditions are known (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). One example of high stringency conditions include hybridization at about 42° C. in 50% formamide, 5×SSC, 5×Denhardt's solution, 0.5% SDS and 100 ug/ml denatured carrier DNA followed by washing two times in 2×SSC and 0.5% SDS at room temperature and two additional times in 0.1×SSC and 0.5% SDS at 42° C.

The term “duplex,” or “duplexed,” as used herein, describes two complementary polynucleotides that are base-paired, i.e., hybridized together.

The term “amplifying” as used herein refers to the process of synthesizing nucleic acid molecules that are complementary to one or both strands of a template nucleic acid. Amplifying a nucleic acid molecule may include denaturing the template nucleic acid, annealing primers to the template nucleic acid at a temperature that is below the melting temperatures of the primers, and enzymatically elongating from the primers to generate an amplification product. The denaturing, annealing and elongating steps each can be performed one or more times. In certain cases, the denaturing, annealing and elongating steps are performed multiple times such that the amount of amplification product is increasing, often times exponentially, although exponential amplification is not required by the present methods. Amplification typically requires the presence of deoxyribonucleoside triphosphates, a DNA polymerase enzyme and an appropriate buffer and/or co-factors for optimal activity of the polymerase enzyme. The term “amplification product” refers to the nucleic acid sequences, which are produced from the amplifying process as defined herein.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

A “plurality” contains at least 2 members. In certain cases, a plurality may have at least 10, at least 100, at least 100, at least 10,000, at least 100,000, at least 10⁶, at least 10⁷, at least or at least 10⁹ or more members.

If two nucleic acids are “complementary”, they hybridize with one another under high stringency conditions. The term “perfectly complementary” is used to describe a duplex in which each base of one of the nucleic acids base pairs with a complementary nucleotide in the other nucleic acid. In many cases, two sequences that are complementary have at least 10, e.g., at least 12 or 15 nucleotides of complementarity.

An “oligonucleotide binding site” refers to a site to which an oligonucleotide hybridizes in a target polynucleotide. If an oligonucleotide “provides” a binding site for a primer, then the primer may hybridize to that oligonucleotide or its complement.

The term “genotyping”, as used herein, refers to any type of analysis of a nucleic acid sequence, and includes sequencing, polymorphism (SNP) analysis, and analysis to identify rearrangements.

The term “sequencing”, as used herein, refers to a method by which the identity of at least 10 consecutive nucleotides (e.g., the identity of at least 20, at least 50, at least 100 or at least 200 or more consecutive nucleotides) of a polynucleotide are obtained.

The term “next-generation sequencing” refers to the so-called parallelized sequencing-by-synthesis or sequencing-by-ligation platforms currently employed by Illumina, Life Technologies, Pacific Bio, and Roche etc. Next-generation sequencing methods may also include nanopore sequencing methods or electronic-detection based methods such as Ion Torrent technology commercialized by Life Technologies.

The term “extending”, as used herein, refers to the extension of a primer by the addition of nucleotides using a polymerase. If a primer that is annealed to a nucleic acid is extended, the nucleic acid acts as a template for extension reaction.

The term “barcode sequence” or “molecular barcode”, as used herein, refers to a unique sequence of nucleotides can be used to a) identify and/or track the source of a polynucleotide in a reaction, b) count how many times an initial molecule is sequenced and c) pair sequence reads from different strands of the same molecule. Barcode sequences may vary widely in size and composition; the following references provide guidance for selecting sets of barcode sequences appropriate for particular embodiments: Casbon (Nuc. Acids Res. 2011, 22 e81), Brenner, U.S. Pat. No. 5,635,400; Brenner et al, Proc. Natl. Acad. Sci., 97: 1665-1670 (2000); Shoemaker et al, Nature Genetics, 14: 450-456 (1996); Morris et al, European patent publication 0799897A1; Wallace, U.S. Pat. No. 5,981,179; and the like. In particular embodiments, a barcode sequence may have a length in range of from 2 to 36 nucleotides, or from 6 to 30 nucleotides, or from 8 to 20 nucleotides.

In some cases, a barcode may contain a “degenerate base region” or “DBR”, where the terms “degenerate base region” and “DBR” refers to a type of molecular barcode that has complexity that is sufficient to help one distinguish between fragments to which the DBR has been added. In some cases, substantially every tagged fragment may have a different DBR sequence. In these embodiments, a high complexity DBR may be used (e.g., one that is composed of at least 10,000 or 100,000, or more sequences). In other embodiments, some fragments may be tagged with the same DBR sequence, but those fragments can still be distinguished by the combination of i. the DBR sequence, ii. the sequence of the fragment, iii. the sequence of the ends of the fragment, and/or iv. the site of insertion of the DBR into the fragment. In some embodiments, at least 95%, e.g., at least 96%, at least 97%, at least 98%, at least 99% or at least 99.5% of the target polynucleotides become associated with a different DBR sequence. In some embodiments a DBR may comprise one or more (e.g., at least 2, at least 3, at least 4, at least 5, or 5 to 30 or more) nucleotides selected from R, Y, S, W, K, M, B, D, H, V, N (as defined by the IUPAC code). In some cases, a double-stranded barcode can be made by making an oligonucleotide containing degenerate sequence (e.g., an oligonucleotide that has a run of 2-10 or more “Ns”) and then copying the complement of the barcode onto the other strand, as described below.

Oligonucleotides that contain a variable sequence, e.g., a DBR, can be made by making a number of oligonucleotides separately, mixing the oligonucleotides together, and by amplifying them en ma se. In other words, the population of oligonucleotides that contain a variable sequence can be made as a single oligonucleotide that contains degenerate positions (i.e., positions that contain more than one type of nucleotide). Alternatively, such a population of oligonucleotides can be made by fabricating them individually or using an array of the oligonucleotides using in situ synthesis methods, cleaving the oligonucleotides from the substrate and optionally amplifying them. Examples of such methods are described in, e.g., Cleary et al (Nature Methods 2004 1: 241-248) and LeProust et al (Nucleic Acids Research 2010 38: 2522-2540).

In some cases, a barcode may be error correcting. Descriptions of exemplary error identifying (or error correcting) sequences can be found throughout the literature (e.g., in are described in US patent application publications US2010/0323348 and US2009/0105959 both incorporated herein by reference). Error-correctable codes may be necessary for quantitating absolute numbers of molecules. Many reports in the literature use codes that were originally developed for error-correction of binary systems (Hamming codes, Reed Solomon codes etc.) or apply these to quaternary systems (e.g. quaternary Hamming codes; see Generalized DNA barcode design based on Hamming codes, Bystrykh 2012 PLoS One. 2012 7: e36852).

In some embodiments, a barcode may additionally be used to determine the number of initial target polynucleotide molecules that have been analyzed, i.e., to “count” the number of initial target polynucleotide molecules that have been analyzed. PCR amplification of molecules that have been tagged with a barcode can result in multiple sub-populations of products that are clonally-related in that each of the different sub-populations is amplified from a single tagged molecule. As would be apparent, even though there may be several thousand or millions or more of molecules in any of the clonally-related sub-populations of PCR products and the number of target molecules in those clonally-related sub-populations may vary greatly, the number of molecules tagged in the first step of the method can be estimated by counting the number of DBR sequences associated with a target sequence that is represented in the population of PCR products. This number is useful because, in certain embodiments, the population of PCR products made using this method may be sequenced to produce a plurality of sequences. The number of different barcode sequences that are associated with the sequences of a target polynucleotide can be counted, and this number can be used (along with, e.g., the sequence of the fragment, the sequence of the ends of the fragment, and/or the site of insertion of the DBR into the fragment) to estimate the number of initial template nucleic acid molecules that have been sequenced.

The terms “sample identifier sequence” or “sample index” refer to a type of barcode that can be appended to a target polynucleotide, where the sequence identifies the source of the target polynucleotide (i.e., the sample from which sample the target polynucleotide is derived). In use, each sample is tagged with a different sample identifier sequence (e.g., one sequence is appended to each sample, where the different samples are appended to different sequences), and the tagged samples are pooled. After the pooled sample is sequenced, the sample identifier sequence can be used to identify the source of the sequences.

The term “strand” as used herein refers to a nucleic acid made up of nucleotides covalently linked together by covalent bonds, e.g., phosphodiester bonds. In a cell, DNA usually exists in a double-stranded form, and as such, has two complementary strands of nucleic acid referred to herein as the “top” and “bottom” strands. In certain cases, complementary strands of a chromosomal region may be referred to as “plus” and “minus” strands, the “first” and “second” strands, the “coding” and “noncoding” strands, the “Watson” and “Crick” strands or the “sense” and “antisense” strands. The assignment of a strand as being a top or bottom strand is arbitrary and does not imply any particular orientation, function or structure. The nucleotide sequences of the first strand of several exemplary mammalian chromosomal regions (e.g., BACs, assemblies, chromosomes, etc.) is known, and may be found in NCBI's Genbank database, for example.

The term “top strand,” as used herein, refers to either strand of a nucleic acid but not both strands of a nucleic acid. When an oligonucleotide or a primer binds or anneals “only to a top strand,” it binds to only one strand but not the other. The term “bottom strand,” as used herein, refers to the strand that is complementary to the “top strand.” When an oligonucleotide binds or anneals “only to one strand,” it binds to only one strand, e.g., the first or second strand, but not the other strand.

The terms “reverse primer” and “forward primer” refer to primers that hybridize to different strands in a double-stranded DNA molecule, where extension of the primers by a polymerase is in a direction that is towards the other primer.

The term “both ends of a fragment”, as used herein, refers to both ends of a double stranded DNA molecule (i.e., the left hand end and the right hand end if the molecule is drawn out horizontally).

The term “the sequence of a barcode”, as used herein, refers to the sequence of nucleotides that makes up the barcode. The sequence of a barcode may be at least 3 nucleotides in length, more usually 5-30 or more nucleotides in length.

The term “or variant thereof”, used herein, refers to a protein that has an amino acid sequence that at least 80%, at least 85%, at least 90%, at least 95%, at least 97%, at least 98% or at least 99% identical to a protein that has a known activity, wherein the variant has at least some of the same activities as the protein of known activity. For example, a variant of a wild type transposase should be able to catalyze the insertion of a corresponding transposon into DNA.

As used herein, the term “PCR reagents” refers to all reagents that are required for performing a polymerase chain reaction (PCR) on a template. As is known in the art, PCR reagents essentially include a first primer, a second primer, a thermostable polymerase, and nucleotides. Depending on the polymerase used, ions (e.g., Mg²⁺) may also be present. PCR reagents may optionally contain a template from which a target sequence can be amplified.

The term “distinguishable sequences” refers to sequences that are different to one another.

The term “target nucleic acid” as use herein, refers to a polynucleotide of interest under study.

The term “target nucleic acid molecule” refers to a single molecule that may or may not be present in a composition with other target nucleic acid molecules. An isolated target nucleic acid molecule refers to a single molecule that is present in a composition that does not contain other target nucleic acid molecules.

The term “region” refers to a sequence of nucleotides that can be single-stranded or double-stranded.

The term “variable”, in the context of two or more nucleic acid sequences that are variable, refers to two or more nucleic acids that have different sequences of nucleotides relative to one another. In other words, if the polynucleotides of a population have a variable sequence, then the nucleotide sequence of the polynucleotide molecules of the population varies from molecule to molecule. The term “variable” is not to be read to require that every molecule in a population has a different sequence to the other molecules in a population.

The term “transposase recognition sequence” refers to a double-stranded sequence to which a transposase (e.g., the Tn5 or Vibhar transposase or variant thereof) binds, where the transposase catalyzes simultaneous fragmentation of a double-stranded DNA sample and tagging of the fragments with sequences that are adjacent to the transposon end sequence (i.e., by “tagmentation”). Transposon end sequences and their use in tagmentation are well known in the art (see, e.g., Picelli et al, Genome Res. 2014 24: 2033-40; Adey et al, Genome Biol. 2010 11:R119 and Caruccio et al, Methods Mol. Biol. 2011 733: 241-55, US20100120098 and US20130203605). The Tn5 transposase recognition sequence is 19 bp in length, although many others are known and are typically 18-20 bp, e.g., 19 bp in length.

The term “adaptor” refers to a nucleic acid that can be joined, via a transposase-mediated reaction, to at least one strand of a double-stranded DNA molecule. As would be apparent, one end of an adaptor may contain a double stranded transposon end sequence. The term “adaptor” refers to molecules that are at least partially double-stranded. An adaptor may be 30 to 150 bases in length, e.g., 40 to 120 bases, although adaptors outside of this range are envisioned.

The term “adaptor-tagged,” as used herein, refers to a nucleic acid that has been tagged by, i.e., covalently linked with, an adaptor. An adaptor can be joined to a 5′ end and/or a 3′ end of a nucleic acid molecule.

The term “tagged DNA” as used herein refers to DNA molecules that have an added adaptor sequence, i.e., a “tag” of synthetic origin. An adaptor sequence can be added (i.e., “appended”) by a transposase.

The term “complexity” refers the total number of different sequences in a population. For example, if a population has 4 different sequences then that population has a complexity of 4. A population may have a complexity of at least 4, at least 8, at least 16, at least 100, at least 1,000, at least 10,000 or at least 100,000 or more, depending on the desired result.

The term “tagmenting” as used herein refers to the transposase-catalyzed combined fragmentation of a double-stranded DNA sample and tagging of the fragments with sequences that are adjacent to the transposon end sequence. Methods for tagmenting are well known as are (see, e.g., Picelli et al, Genome Res. 2014 24: 2033-40; Adey et al, Genome Biol. 2010 11:R119 and Caruccio et al, Methods Mol. Biol. 2011 733: 241-55, US20100120098 and US20130203605). Kits for performing tagmentation are commercially sold by a variety of manufacturers.

The term “transposase complex” refers to a complex that contains a transposase (which typically exists as a dimer of transposase polypeptides) that is bound to i) a first adapter molecule, wherein the first adapter molecule comprises at least a recognition sequence for the transposase, and ii) a second adaptor molecule, wherein the second adapter molecule comprises at least a recognition sequence for the transposase.

The term “loaded” refers to a process by which a transposase and molecule containing a transposon end sequence are mixed together to form complexes that contain the transposase bound to the molecule.

The term “filling in” refers to a reaction in which a single-stranded region, e.g., a 5′ overhang, is filled in by the action of a polymerase, e.g., a non-strand displacing or strand displacing polymerase.

The term “same barcode on both strands” and grammatical equivalents thereof refers to a double stranded molecule that has a barcode sequence covalently linked at the 5′ end of one strand and the complement of the barcode sequence covalently linked at the 3′ end of the other strand.

The term “collectively comprise” refers to the types of molecules that are found in a population of transposase complexes as a whole, rather than individual transposase complexes.

The term “collectively hybridize to” refers to the attributes of a population of primers as a whole A population of primers that are capable of priming DNA synthesis from a variable sequence have a sequence of at least 6, at least 8 or at least 10 nucleotides at the 3′ end that is complementary to a variable sequence such the primers hybridize to and prime DNA synthesis from all of the variable sequences, or complements thereof. For example, a set of primers that collectively hybridize to and are capable of priming DNA synthesis from three different adaptor sequences has three primers, where each of the six primers has a 3′ end sequence that hybridizes to (i.e., is complementary to) and primes DNA synthesis from a different adaptor sequence.

The term “of the formula” means that the individual molecules in a population are described by, i.e., encompassed by, the formula.

Certain polynucleotides described herein may be referred by a formula (e.g., “X-Y”). Unless otherwise indicated the polynucleotides defined by a formula is oriented in the 5′ to 3′ or 3′ to 5′ direction. The components of the formula, e.g., “X”, “Y”, etc., refer to separately definable sequences of nucleotides within a polynucleotide, where, unless implicit from the context, the sequences are linked together covalently such that a polynucleotide described by a formula is a single molecule. In some cases the components of the formula are immediately adjacent to one another in the single molecule. Unless otherwise indicated or implicit from the context, a polynucleotide defined by a formula may have additional sequence, a primer binding site, a molecular barcode, a promoter, or a spacer, etc., at its 3′ end, its 5′ end or both the 3′ and 5′ ends. As would be apparent, the various component sequences of a polynucleotide (e.g., X, Y, etc, etc.,) may independently be of any desired length as long as they capable of performing the desired function (e.g., hybridization to another sequence). For example, the various component sequences of a polynucleotide may independently have a length in the range of 8-80 nucleotides, e.g., 10-50 nucleotides or 12-30 nucleotides.

Other definitions of terms may appear throughout the specification.

### DESCRIPTION OF EXEMPLARY EMBODIMENTS

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, the some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to herein are expressly incorporated by reference.

Provided herein are various compositions, methods and kits for tagging samples containing double stranded DNA molecules. The compositions, methods and kits can be employed to analyze genomic DNA from virtually any organism, including, but not limited to, plants, animals (e.g., reptiles, mammals, insects, worms, fish, etc.), tissue samples, bacteria, fungi (e.g., yeast), phage, viruses, cadaveric tissue, archaeological/ancient samples, etc. In certain embodiments, the genomic DNA used in the method may be derived from a mammal, wherein in certain embodiments the mammal is a human. In exemplary embodiments, the sample may contain genomic DNA from a mammalian cell, such as, a human, mouse, rat, or monkey cell. The sample may be made from cultured cells or cells of a clinical sample, e.g., a tissue biopsy, scrape or lavage or cells of a forensic sample (i.e., cells of a sample collected at a crime scene). In particular embodiments, the nucleic acid sample may be obtained from a biological sample such as cells, tissues, bodily fluids, and stool. Bodily fluids of interest include but are not limited to, blood, serum, plasma, saliva, mucous, phlegm, cerebral spinal fluid, pleural fluid, tears, lactal duct fluid, lymph, sputum, synovial fluid, urine, amniotic fluid, and semen. In particular embodiments, a sample may be obtained from a subject, e.g., a human.

In some embodiments, the sample comprises DNA fragments obtained from a clinical sample, e.g., a patient that has or is suspected of having a disease or condition such as a cancer, inflammatory disease or pregnancy. In some embodiments, the sample may be made by extracting fragmented DNA from an archived patient sample, e.g., a formalin-fixed paraffin embedded tissue sample. In other embodiments, the patient sample may be a sample of cell-free circulating DNA from a bodily fluid, e.g., peripheral blood. The DNA fragments used in the initial steps of the method should be non-amplified DNA that has not been denatured beforehand. In other embodiments, the DNA in the sample may already be partially fragmented (e.g., as is the case for FFPE samples and circulating cell-free DNA (cfDNA), e.g., ctDNA). The method finds particular use in the analysis of unamplified genomic DNA.

In some embodiments, the amount of DNA in the sample may be limiting. For example, the initial sample of DNA may contain less than 200 ng of fragmented DNA, e.g., 10 pg to 200 ng, 100 pg to 200 ng, 1 ng to 200 ng or 5 ng to 50 ng, or less than 10,000 (e.g., less than 5,000, less than 1,000, less than 500, less than 100 or less than 10) haploid genome equivalents, depending on the genome.

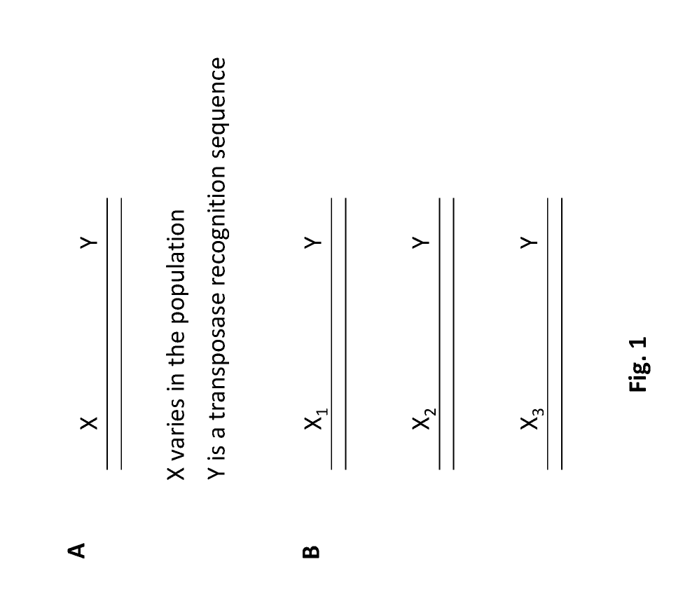

As noted above, in some embodiments, the method may comprise tagmenting the nucleic acid sample with a population of transposase complexes. Each transposase complex comprises a dimer of a transposase, and a pair of adaptors. Collectively, the population of transposase complexes may comprise: i. a transposase (which is usually in the form of a dimer of transposase polypeptides) and ii. a set of adaptors of the formula X-Y, wherein: region X is a variable sequence (e.g., a sequence that is of at least 6, at least 8, or at least 10 nucleotides in length) that has a complexity of n, wherein n is at least 3 (e.g., at least 4, at least 5, at least 6, at least 7, at least 8, at least 9, or at least 10, such as in the range of 3 to 100, 4 to 50, 5 to 40 or 6 to 30), and region Y is a recognition sequence for the transposase, i.e., a double-stranded transposase recognition sequence. One example of an adaptor that can be used in the method is illustrated in FIG. 1. Panel A of FIG. 1 illustrates an example of an embodiment of a set of adaptors, where region Y is a transposase recognition sequence and region X varies in sequence. As shown, region X can be double stranded. However, in some embodiments, X can be single stranded (e.g., may be part of a 5′ overhang). Panel B illustrates a set of adaptors that has three members. In this embodiment, n=3, although, as noted above, n can be a larger integer. As shown, the different sequences of region X (X₁, X₂ and X₃) are different sequences. The different sequences of region X are chosen to provide specific priming such that a primer that has a 3′ end that hybridizes to and primes from one sequence of X (e.g., X₁), does not hybridize to or prime from another sequence of X (e.g., X₂ or X₃), etc. This tagmentation step produces a collection of fragments that are tagged with the variable sequences (e.g., X₁, X₂, X₃, up to Xₙ). Depending on how the tagmentation is done, for example varying the stoichiometric ratio of transposase enzyme to nucleic acids, the tagged fragments may have a median size that is below 1 kb (e.g., in the range of 50 bp to 1000 bp, or 80 bp to 400 bp), although fragments having a median size outside of this range may be used.

Next, the method comprises amplifying the tagged fragments, by PCR, using a set of primers, wherein the set of primers comprises at least n different primers (i.e., at least the same number primers as the number of different X sequences) and the n different primers collectively hybridize to all of the different sequences of region X, or a complement thereof. Specifically, a first primer of the n primers will hybridize to and prime synthesis from a first sequence of region X (e.g., sequence X₁), a second primer of the n primers will hybridize to and prime synthesis from a second sequence of region X (e.g., sequence X₂), a third primer of the n primers will hybridize to and prime synthesis from a third sequence of region X (e.g., sequence X₃) and a fourth primer of the n primers will hybridize to and prime synthesis from a fourth sequence of region X (e.g., sequence X₁), and so on. This step results in the production of amplification products. As would be apparent, the variable sequences of the adaptors should not be in the genomic DNA being analyzed, and the primers should be designed to hybridize to a sequence of the variable region of the adaptor, rather than to the genomic DNA.

Because the tagmentation step uses adaptors that have a variable sequence and the amplification step uses primers that hybridize to the variable sequence in the adaptor, use of the method provides a greater representation of a genome in the amplification products. This is because the tagmentation step results in a larger number of asymmetrically-tagged fragments, (i.e., fragments that have a different adaptor sequence at each end, or, more specifically, fragments in which the adaptor sequence at the 5′ end of the top strand is not complementary to the adaptor sequence at the 3′ end of the top strand) relative to methods that rely on one or two adaptors. Because, in the present method, more fragments are asymmetrically-tagged, the entire population of fragments can be more efficiently amplified.

The transposon recognition sequence (also known as a transposon end sequence) used in the method is a double-stranded sequence to which the transposase (e.g., a Tn5 or Vibhar transposase, or variant thereof) binds. The Tn5 transposon recognition sequence is 19 bp in length (see, e.g., Vaezeslami et al, J. Bacteriol. 2007 189 20: 7436-7441), although many others are known and in some cases may be 18-20 bb. In this method, the transposase complex comprises a transposase loaded with two adaptor molecule that each contain a recognition sequence for the transposase at one end. The transposase catalyzes simultaneous fragmentation of the sample tagging of the fragments with sequences that are adjacent to the transposon recognition sequence (i.e., “tagmentation”). In some cases, the transposase enzyme can insert the nucleic acid sequence into the polynucleotide in a substantially sequence-independent manner. The transposase can be prokaryotic, eukaryotic or from a virus. This initial step of the method may be done by loading a transposase with oligonucleotides that have been annealed together so that at least the transposase recognition sequence is double stranded. The adaptors used in the method are typically made of oligonucleotides that have been annealed together.

In some embodiments, the transposase complexes may each comprises a pair of the same adaptor. In these embodiments, the transposase may be loaded with the adaptors in different containers, such that each transposase is loaded with two molecules of the same adaptor. The different transposase complexes can be pooled prior to tagmentation. Use of transposase complexes that each comprise a pair of the same adaptor allows one to perform contiguity-preserving transposition sequencing, as described in Adel et al (Genome Res. 2014 24: 2041-9), Amini et al (Nat Genet. 2014 46: 1343-9), Christiansen et al (Methods Mol Biol. 2017 1551: 207-221) and US907425 1. In other embodiments, the population of transposase complexes may comprise transposase complexes in which the adaptors are different. In these embodiments, the different adaptors may be made in different vessels (e.g., by annealing oligonucleotides together), pooling the adaptors, and loading the pooled adaptors onto a transposase in a single reaction. In this method, most transposase complexes will contain two different adaptors, and the portion of transposase complexes that contain two different adaptors increases with the complexity of the variable sequence of the adaptors.

As noted above, in some cases, region X may at least partially single-stranded. In these embodiments, the single-stranded part of region X may contain a molecular barcode, e.g., a barcode that a) identifies and/or tracks the source of a polynucleotide in a reaction, b) may be used to count how many times an initial molecule is sequenced, c) correct sequencing errors and/or d) may be used to pair sequence reads from different strands of the same molecule, as described above. In some cases, this sequence may be a random sequence, although any variable sequence can be used in some cases. In these embodiments, the single stranded region may be filled in after tagmentation (e.g., by extending the genomic DNA using the single stranded region as a template), thereby copying the barcode onto both strands. As such, in some embodiments, the method may comprise making the ends of the fragments produced in the tagmentation step double stranded prior to amplification, thereby adding the barcode to both strands of the fragment.

In certain embodiments, the method may further comprise sequencing at least some of the amplification products. As would be apparent, in these embodiments, the adaptors used may contain sequences that are compatible with use in the sequencing platform being used for sequencing, where those sequences are downstream from the variable sequences. Alternatively, the primers used for amplification step may contain 5′ tails containing sequences that are compatible with use in the sequencing platform being used for sequencing or, the amplification products themselves may be amplified using primers that contain 5′ tails containing sequences that are compatible with use in the sequencing platform being used for sequencing. The products may be sequenced using any suitable method including, but not limited to Illumina's reversible terminator method, Roche's pyrosequencing method (454), Life Technologies' sequencing by ligation (the SOLiD platform), Life Technologies' Ion Torrent platform, nanopore sequencing or Pacific Biosciences' fluorescent base-cleavage method. Examples of such methods are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009; 513:19-39) English (PLoS One. 2012 7: e47768) and Morozova (Genomics. 2008 92:255-64), which are incorporated by reference for the general descriptions of the methods and the particular steps of the methods, including all starting products, reagents, and final products for each of the steps. In some embodiments, the sequencing may be done by paired end sequencing.

In some embodiments, the tagged DNA may be sequenced using nanopore sequencing (e.g., as described in Soni et al. Clin. Chem. 2007 53: 1996-2001, or as described by Oxford Nanopore Technologies). Nanopore sequencing is a single-molecule sequencing technology whereby a single molecule of DNA is sequenced directly as it passes through a nanopore. A nanopore is a small hole, of the order of 1 nanometer in diameter. Immersion of a nanopore in a conducting fluid and application of a potential (voltage) across it results in a slight electrical current due to conduction of ions through the nanopore. The amount of current which flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence. Nanopore sequencing technology is disclosed in U.S. Pat. Nos. 5,795,782, 6,015,714, 6,627,067, 7,238,485 and 7,258,838 and U.S. Pat Appln Nos. 2006003171 and 20090029477.

The sequencing step results in a plurality sequence reads, e.g., at least 10,000, at least 50,000, at least 100,000, at least 500,000, at least 10M at least 10M at least 100M or at least 1B sequence reads. In some cases, the reads are paired-end reads. The sequence reads can be analyzed to identify sequence variations in the sample, to provide a copy number analysis or for de novo sequence assembly, for example.

Depending on how the method is performed, in some embodiments the sequence reads may each comprise i. the sequence of at least part of the sequence of a DNA fragment and ii. the sequence of at least part of a primer used for amplification, and/or a molecular barcode.

The sequence of the primer or molecular barcode sequence may be identified in the sequence reads, and used to identify sequence errors, for allele calling, for assigning confidence, to perform copy number analysis, duplex sequencing and to estimate gene expression levels using methods that can be adapted from known methods, see, e.g., Casbon (Nucl. Acids Res. 2011 39: e81), Fu (Proc. Natl. Acad. Sci. 2011 108: 9026-9031) and Kivioia (Nat. Methods 2011 9: 72-74). If an error correctable barcode is used, then such analyses may become more accurate because, even if one barcode is mis-read, the error can be corrected or the read can be eliminated.

The sequence reads may be processed and grouped in any convenient way. In some embodiments, the sequence reads may be grouped by the primer sequence and/or barcode and, optionally, by one or more of the fragmentation breakpoints of the sequence read, where a fragmentation breakpoint is represented by the “end” of the sequence after the tags have been trimmed off. Assuming fragmentation is random, or semi-random, different fragments having the same sequence can be distinguished by their fragmentation breakpoints. Different fragments that have the same fragmentation breakpoints can be further distinguished by the primer sequence and/or barcode. Grouping the sequence reads by their fragmentation breakpoints and their primer sequence and/or barcode provides a way to determine if a particular sequence (e.g., a sequence variant) is present in more than one starting molecule. In some implementations, initial processing of the sequence reads may include identification of molecular barcodes (including sample identifier sequences or sub-sample identifier sequences), and/or trimming reads to remove low quality or adaptor sequences. In addition, quality assessment metrics can be run to ensure that the dataset is of an acceptable quality. In some embodiments therefore, the method may comprise identifying identical or near-identical sequence reads that have identical or near-identical fragmentation breakpoints but different primer sequences and/or barcode sequences. In these embodiments, sequence reads derived from two fragments that are otherwise near identical in sequence and fragmentation breakpoints can be distinguished by their primer sequence. As would be apparent, the confidence that a potential sequence variation is a true variation (rather than a PCR or sequencing error) increases if it is present in more than one molecule. Likewise, copy number variations can be measured more accurately if one can distinguish fragments that are otherwise identical to one another.

Molecules that contain identical or near-identical fragmentation breakpoints have the same 5′ end, the same 3′ end, or the same 5′ and 3′ ends, where any differences are due to a PCR error, sequencing error, mapping or alignment error or somatic mutation. A fragmentation breakpoint can be determined by removing the adaptor sequence from a sequence read, leaving the sequence of the target. The first nucleotide of the trimmed sequence represents the first nucleotide after the fragmentation breakpoint. In sequencing an amplified sample, two sequence reads that correspond to fragments that have identical or near-identical fragmentation breakpoints can be derived from the same initial fragment. In many cases, 8-30 nucleotides at the end of a trimmed sequence can be compared to the ends of other trimmed sequences to determine if the fragmentation breakpoints are the same or different. In many cases, fragmentation breakpoints can be identified after mapping reads to a reference sequence. After mapping fragmentation breakpoints may be identified using software e.g., Picard MarkDuplicates (available from the Broad Institute), Samtools rmdup (see, e.g., Li et al. Bioinformatics 2009, 25: 2078-2079) and BioBamBam (Tischler et al, Source Code for Biology and Medicine 2014, 9:13).

As would be recognized, many of the analysis steps of the method, e.g., sequence trimming, grouping, sequence assembly, variant identification, copy number analysis etc., can be implemented on a computer. In these embodiment, the sequence reads may be analyzed by a computer and, as such, instructions for performing the steps set forth below may be set forth as programming that may be recorded in a suitable physical computer readable storage medium.

In certain embodiments, a general-purpose computer can be configured to a functional arrangement for the methods and programs disclosed herein. The hardware architecture of such a computer is well known by a person skilled in the art, and can comprise hardware components including one or more processors (CPU), a random-access memory (RAM), a read-only memory (ROM), an internal or external data storage medium (e.g., hard disk drive). A computer system can also comprise one or more graphic boards for processing and outputting graphical information to display means. The above components can be suitably interconnected via a bus inside the computer. The computer can further comprise suitable interfaces for communicating with general-purpose external components such as a monitor, keyboard, mouse, network, etc. In some embodiments, the computer can be capable of parallel processing or can be part of a network configured for parallel or distributive computing to increase the processing power for the present methods and programs. In some embodiments, the program code read out from the storage medium can be written into memory provided in an expanded board inserted in the computer, or an expanded unit connected to the computer, and a CPU or the like provided in the expanded board or expanded unit can actually perform a part or all of the operations according to the instructions of the program code, so as to accomplish the functions described below. In other embodiments, the method can be performed using a cloud computing system. In these embodiments, the data files and the programming can be exported to a cloud computer that runs the program and returns an output to the user.

Further details of some implementations of the method may be described in greater detail below.

This disclosure provides, among other things, a way to improve the efficiency of transposase-mediated “tagmentation” methods using multiple PCR primers (>2) to amplify the tagmented products. The method can incorporate the use of molecular barcoding by tagmentation while eliminating the need for enzymatic whole genome amplification. The method can be applied to sample barcoding, molecular barcoding and phasing of adjacent paired-end reads from the same target DNA duplex (for haplotype sequencing). This assay may also be compatible with duplex sequencing, where both the forward and reverse DNA strands are tagged with the same molecular barcode. This phasing approach is enabled by loading transposases with tagged oligos independently, such that both tranposases of each molecular dimer incorporate the same barcode or index.

The transposase recognition sequence of the adaptor may be the transposase recognition sequence of a Tn transposase (e.g. Tn3, Tn5, Tn7, Tn10, Tn552, Tn903), a MuA transposase, a Vibhar transposase (e.g. from Vibrio harveyi), although the transposase recognition sequence for other transposases (Ac-Ds, Ascot-1, Bs1, Cin4, Copia, En/Spm, F element, hobo, Hsmar1, Hsmar2, IN (HIV), IS1, IS2, IS3, IS4, IS5, IS6, IS10, IS21, IS30, IS50, IS51, IS150, IS256, IS407, IS427, IS630, IS903, IS911, IS982, IS1031, ISL2, L1, Mariner, P element, Tam3, Tc1, Tc3, Tel, THE-1, Tn/O, TnA, Tn3, Tn5, Tn7, Tn10, Tn552, Tn903, Tol1, Tol2, TnlO, Tyl, including variants thereof) can also be used. Transposase is unique among enzymes in that it works as a dimer of transposase protein molecules to make a double-stranded breaks in the target DNA and ligate two short cargo DNA duplexes onto the 5′-ends of both of the cut target duplex. The result is that the 5′-end of each cut end of the DNA duplex is tagged with a single-strand of DNA carried by the transposase dimer.

The basic principle of an embodiment of the method is the use of transposases loaded with different distinct oligos with distinct primer binding sites for use with a set of more than two distinct primer sequences. In conventional methods, only two primers sequences are used for PCR amplification of DNA for fragments that have been ligated at both ends with oligonucleotides by transposition by transposase. In those cases, then at best about half of the transposed fragmented DNA is sequenceable. This is because the transposition process randomly adds either of the two sequences complementary to two primers used. Whenever a fragment is tagmented with the same primer binding sequence at both ends, after a first extension sequence or the first cycle of PCR both ends of each strand of the product are complementary, forming a large hairpin, and amplification by PCR is suppressed. For an equal mixture of transposases loaded with each of two primers, the best possible case is the production of fragments where 50% of them are amplifiable.

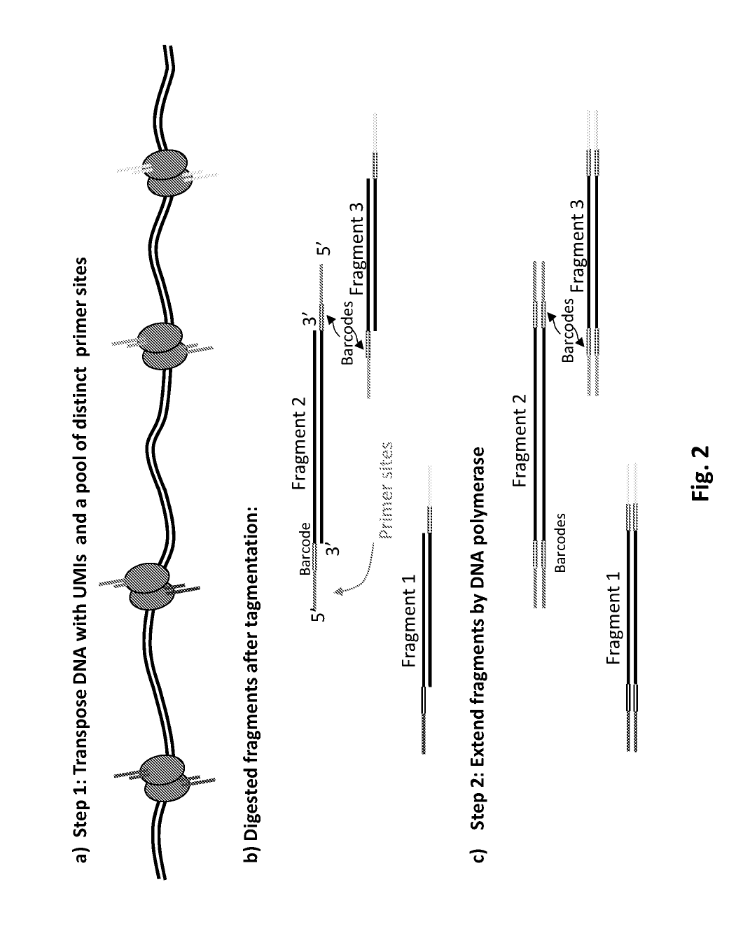

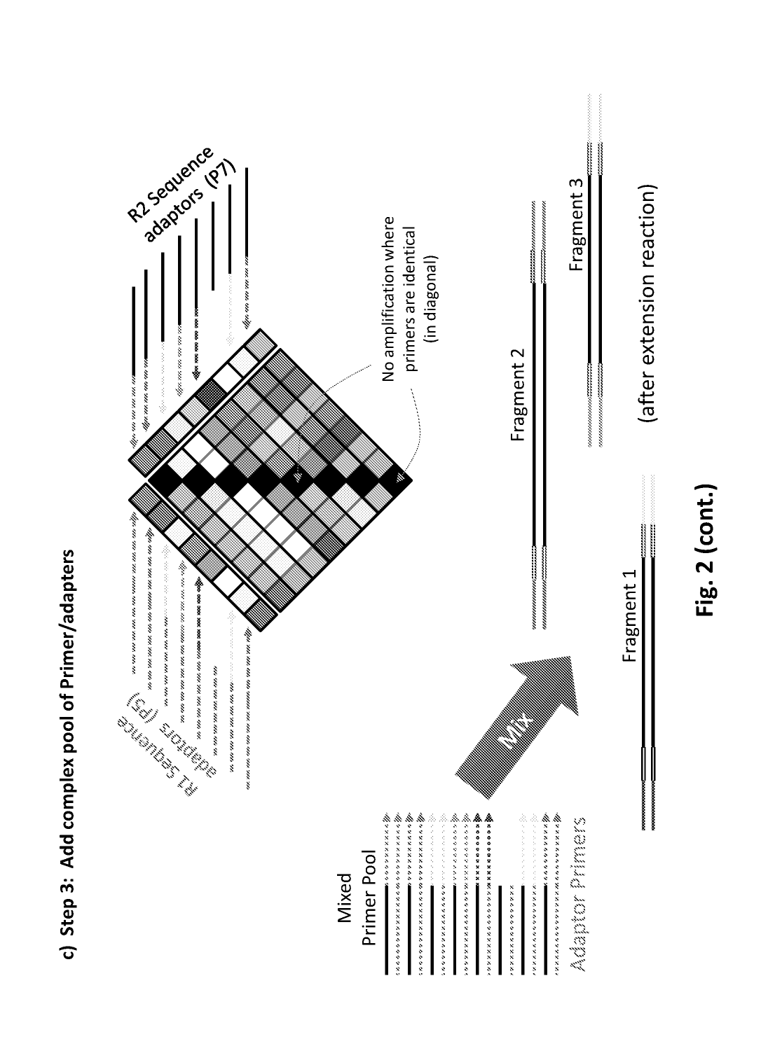

Some features of an implementation of the method are depicted in FIG. 2. In this implementation, the first step of the method comprises reacting genomic DNA or double stranded DNA is exposed to a transposase enzyme mix, where different transposase dimers are loaded with different combinations of sequence adaptors and priming sequences. In some embodiments, the transposases are pre-loaded with individual sequence duplexes individually (such that each complex comprises two of the same adaptor), and in other embodiments, the sequences are pooled before loading the transposase (such that each complex has a high likelihood of containing two different adaptors). The loading process determines whether both transposase molecules of the dimer are loaded identically. In this implementation, the DNA fragments produced by transposition may have 5′ overhangs on both ends with sequences ligated by the transposase. These include a double stranded transposase binding sequence, and a 5′ overhang containing a molecular barcode (or index sequence) and a primer binding sequence or a sequencing adaptor.

After ligation, the 3′ end can be extended by a DNA polymerase to make a copy of the overhang, as shown in FIG. 2. This extension can be done as a separate reaction or immediately prior to the first denaturation step in a PCR reaction.

FIG. 2 also depicts a scheme for combining primer binding sequences using sequencing adaptors for amplification. As depicted in FIG. 2 (in different colors) if 8 distinct primer binding sequences are loaded into transposases (with or without sequencing adaptors attached), only those fragments with distinct ends will be amplified by PCR, and those where they are the same will be suppressed by the formation of stable hairpin loops. If the adaptor sequences are added directly throughout the whole process, then all amplification products will have adaptors at both ends, but only a subset (as high as 50%) will have both distinct adaptors. If there were N distinct primers, then about (N−1)/N of the original DNA fragments will be sequenceable and 1/N will be unsequenceable (assuming an equal mixture of all primer sequences and uniform amplification across primers). For this reason, more distinct primer binding sequences is better than fewer.

The primers used for amplification do not need to have sequencing adaptor sequences (e.g., P5 and P7 sequences) attached. Such can be added either throughout the PCR amplification, or late in the process. In one embodiment, the first K cycles of PCR are performed with short primer sequences without sequencing adaptors, and the later J, typically 2-4, cycles amplification with longer exogenous primer sequences that have adaptor sequences at their 5′ ends. In this way the sequencing adaptors are not extended during the bulk of the PCR, but most amplified material will become sequenceable by the last few PCR cycles. Alternatively, sequencing adaptors can be added by ligation after PCR. These latter embodiments may mean that 50% of the final PCR products are unsequenceable, but most of the original material is amplified multiple times and is hence represented in the sequenceable half of the product.

There are several methods for adding sequencing adaptor sequences onto amplification products while those products are being amplified. For example, in one approach the first set of primers are introduced in limited, but identical, quantities, and simply keep cycling for a few extra cycles, but run out once all the primers are consumed. Then, add the longer adaptor primers and continue for several more PCR cycles. Another approach is to increase the efficiency of binding of the second stage primers so that they out-compete the original primer set. This can be done by lengthening the primer sequence, and (optionally) increasing the temperature of the annealing phase of the thermal cycling. Finally another approach is to bead purify the products so that longer DNA products are separated from shorter primers. These approaches can be applied individually, or used in combination. Other approaches would be apparent

For applications involving small quantities of genomic DNA, where the sequencing depth exceeds the number of cells, then the genomes of each cell are oversampled with sufficient redundancy to provide error suppression or reduction redundancy to eliminate errors introduced by amplification or sequencing. For tumor or cancer samples, somatic variants are common and need to be differentiated from other sources of noise.

One of the greatest challenges in single-cell sequencing and in the clonal analysis of tumor samples is the detection and identification of both alleles of every cell or every clonal population of a diploid sample. For a biallelic sample, the absence of information of one allele of the other is called an allelic dropout, or ADO. ADOs are caused by insufficient coverage, or biases in the amplification of the original DNA that lead to allelic imbalances in the allelic sequences detected. To minimize ADO it is useful to maximize the detection efficiency of the assay. In conventional transposase assays two distinct sequences are loaded into transposase and ligated onto the ends of the DNA fragmented by the transposase. When the opposite ends are sequenced, they must have two distinct adaptor sequences, used for amplification in “polonies” on the sequencer.

In many single-cell or low-input assays that use transposase, the first step is whole genome amplification (WGA). The best enzymes for WGA are with high processivity, strand-displacement activity and proofreading activity that embellishes them with low error rates, including, for example Phi29 and Bst. Assays for WGA include multiple displacement amplification (MDA) and Omniplex. In the assay described here, no amplification is necessary before the cleavage and tagging of the DNA by transposase. Instead the amplification follows the transpose digest step. The first stage of amplification is accomplished by the use of a mixture of primers. In this method the transposases are loaded with two or more primer sequences. The more distinct primer sequences used, the more efficient the assay can be in terms of coverage of the target DNA. During PCR amplification multiple primers are used and only those that match the two ends of the target duplex, and are distinct from each other. In some embodiments the primer sequences are ligated onto the end left by the transposase.

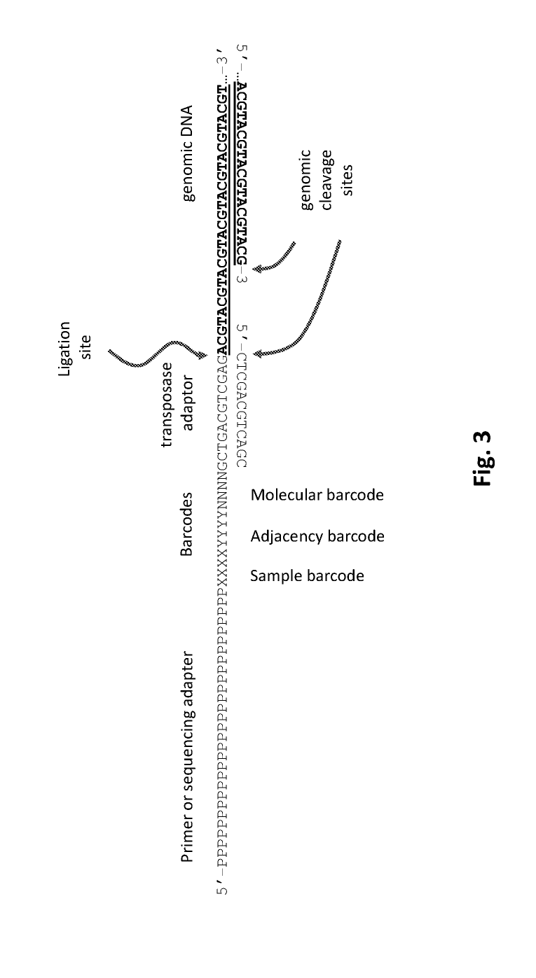

FIG. 3 shows one possible construction for the adaptor used in adjacency barcoding (or contiguity-preserving transposition sequencing). The transposase can be loaded with a duplex that is short on one strand and has an overhang on the other. The double stranded 19-bp end region, which is recognized by the transposase enzyme, is kept minimal to prevent other transposases from attacking it during the tagmentation assay. There are numerous sequences that are recognized by transposases. Biologically, the end sequences are different at the two ends of an insert (called OE and IE, depending on whether they are on the inside or the outside of an inserted region). The recognition sequence for the Vibrio harveyi is illustrated. The 5′-end of the 5′-overhang, as drawn in the figure, has a PCR primer sequence, a sequencing adapter, or both. The barcode sequences are between the recognition sequence and the primer sequence. Shown are several bases for the sample barcode, several bases for the adjacency barcode, and several more (probably unnecessary) for molecular barcode. These elements can be arranged in any order and do not need to be contiguous with one another. The number of bases use in each barcode depends on factors like the number of samples to be used, the number of molecules to be differentiated and the level of redundancy desired in the barcodes. Molecular barcodes are often produced by means of synthesizing “degenerate bases”, meaning that any canonical base can be incorporated at any position within the barcode sequence.

This design provides a way by which both strands of a double-stranded molecule can be tagged with the same barcode such that, after sequencing or amplification, the sequence reads derived from the top strand can be linked and/or compared to the sequence reads derived from the bottom strand. This feature is significant because “real” mutations should be in both strands (i.e., in both the top strand and the bottom strand), and knowing whether a sequence read is from the top strand or the bottom strand allows the top strand sequences to be compared with bottom strand sequences to provide more confidence that a variation in a sequence really corresponds to a mutation.

In some embodiments, the sequencing adaptors and/or amplification primer sites can be added to the barcoded target DNA duplexes by ligation. In this case, the most specific way to perform the ligation is to design the barcoded adapter sequences with an overhang. In this way the amplification priming sequences can be added as pre-hybridized duplexes with a complementary overhang that ligates specifically to the transposase-adapted ends of the target DNA duplexes. In this embodiment the pool of adapted sequences should include a mix of multiple sequences complementary to the pool of primer sequences to be added before PCR.

Molecular barcoding is the ligation of short but unique sequences to each original target molecules, before amplification, and in a manner that preserves the identity of the original molecule even after amplification. The method is useful to filter out errors made during copying and amplifying the DNA. This type of barcoding is done using complex pools of DNA duplexes, with sufficiently complexity to identify the original molecule. These pools of complex barcodes can either be created deterministically, by directly synthesizing each barcode directly, or more efficiently, using a run of degenerate bases (e.g., a run of Ns).

Molecular barcoding is commonly applied to the sequencing of messenger RNA (mRNA) than genomic DNA. This may be because the molecular barcodes can easily be added to the first cDNA sequence during the reverse transcription process as demonstrated by a number of studies. [Refs: Islam et al. 2014, Fan et al., Klein et al. Cell “Droplet Barcoding for Single-Cell Transcriptomic Applied to Embryonic Stem Cells”, Cell (2015), Macosco et al. “Highly Parallel Genome-wide Expression Profiling of Individual Cells Using Nanoliter Droplets”, Cell (2015)]. Molecular barcoding with a transposase-sequencing assay has been applied to mRNA.

The present method should be compatible with duplex sequencing. Duplex sequencing is a barcoding approach that has been used to reduce sequencing noise by taking advantage of the redundancy of the original genomic molecules given by the fact that the forward and reverse strands are complementary. In principle, the method the works by ligating different molecular barcodes to the forward and reverse strands, in such a way such that the two original strands can be distinguished, even after amplification, or upon sequencing. As practiced by Kennedy et al. (Nat Protoc. 2014 9, 2586-2606) and Schmidt et al. (PNAS, 2012 109, 14508), two distinct double-stranded barcodes are ligated to opposite ends of each double-stranded DNA fragment and the ligated duplex contains different sequencing adapters on the two strands using a Y-adaptor. In this method, the ligation may be done by the transposase instead of a ligase.

Applying molecular barcodes to genomic DNA sequencing with tagmentation by transposase is straightforward by eliminating the whole genome amplification step used in the usual genomic workflow and by incorporating molecular barcodes into duplexes loaded into the transposases. In some assays the sample barcodes on the 5′-overhang sequence of the duplex cargo of the transposase molecules. This overhang is a good place to incorporate either molecular barcodes or the adjacency barcodes of this method.

Once molecular barcodes are added to the conventional sample-barcoded transposase assay, duplex sequencing can be performed as well. In conventional methods, a “repair step” can be used to fill-in step and replace the 9 nucleotides removed at the 3′ ends of the target sequences by the transposase. This repair step is performed via a 68° C. extension step for 2 minutes just prior to the 98° C. denaturation initiation of the PCR amplification. This repair step can also make the single stranded 5′ end of the present adaptor double stranded.

Additional requirements for duplex sequencing are associated with making sure that the assay has sufficient sequencing depth and uniformity that both source strands are detected with the same (or complementary pair of) distinct molecular barcodes. And, finally, the additional analysis is required to use these independent detection events for error correction or error reduction. That additional analysis is for the purposes of filtering so that only those variant allelic sequences for which both the forward and reverse strands with the same molecular barcodes are consistent for the variant allele. This means both sufficient depth and strand balances that both strands are likely to be well represented in the reads.

Finally, the present method is compatible with target enrichment methods using DNA or RNA capture probes, such as Agilent's SureSelect products.

In certain embodiments, the sample sequenced may comprise a pool of nucleic acids from a plurality of samples, wherein the nucleic acids in the sample have a different molecular barcode to indicate their source. In some embodiments, the nucleic acids being analyzed may be derived from a single source (e.g., from different sites or a time course in a single subject), whereas in other embodiments, the nucleic acid sample may be a pool of nucleic acids extracted from a plurality of different sources (e.g., a pool of nucleic acids from different subjects), whereby “plurality” is meant two or more. As such, in certain embodiments, a nucleic acid sample can contain nucleic acids from 2 or more sources, 3 or more sources, 5 or more sources, 10 or more sources, 50 or more sources, 100 or more sources, 500 or more sources, 1000 or more sources, 5000 or more sources, up to and including about 10,000 or more sources. These molecular barcodes allow the sequences from different sources to be distinguished after they are analyzed. Such barcodes may be in the adaptor, or they may be added the amplification process (after tagging).

This method can be applied to any genomic or mitochondrial DNA from mammals, plants, bacteria, fungi or Achaea. Useful applications of this method relate to cancer diagnostics and cancer research, such as cancer etiology, for example in elucidating tumor development, metastatic processes or clonal evolution or drug evasion. Determining the clonal composition of a tumor necessitates the detection of numerous somatic variants and determining the associations between those variants amongst the various clones and subclones of a tumor. The vast majority of somatic variants in tumors are heterozygous in diploid genomes. The association with somatic variants with heterozygous SNPs is useful both for associating clones and subclonal data as well as being helpful in error detection and correction.

Also provided are compositions that comprise a population of transposase complexes. In these embodiments, the population of transposase complexes comprise: i. a transposase; and ii. a set of adaptors of the formula X-Y, wherein: region X is a variable sequence that has a complexity of n, wherein n is at least 3, and Y is a double-stranded recognition sequence for the transposase. These transposase complexes are present in the same container as a mix. Variations of this composition may be indicated by the foregoing description. For example, as described above, the transposase may be a Tn5 or Vibhar transposase, region X is at least partially single stranded and may contain a molecular barcode, and n may be in the range of 5 to 40.

### Kit

Also provided by this disclosure are kits for practicing the subject method, as described above. In certain embodiments, the kit may comprise (a) a transposase, (b) a set of adaptors of the formula X-Y, wherein: region X is a variable sequence that has a complexity of n, wherein n is at least 3, and region Y is a double-stranded recognition sequence for the transposase, (c) a set of primers, wherein the set of primers comprises at least n different primers and the n different primers collectively hybridize to each of the different sequences of region X, or a complement thereof. This kit can be implemented in a variety of different ways. For example, the set of adaptors are present as a mixture in the same container, the adaptors in each set have the same sequence X, and each set of adaptors is present in a different container, the transposase is a Tn5 or Vibhar transposase, region X is at least partially single stranded, the single stranded part of region X comprises a molecular barcode and/or n is in the range of 5 to 40.

Either of the kits may additionally comprise suitable reaction reagents (e.g., buffers etc.) for performing the method. The various components of the kit may be present in separate containers or certain compatible components may be precombined into a single container, as desired. In addition to the reagents described above, a kit may contain any of the additional components used in the method described above, e.g., one or more enzymes and/or buffers, etc.

In addition to above-mentioned components, the subject kits may further include instructions for using the components of the kit to practice the subject methods, i.e., to instructions for sample analysis. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### EMBODIMENTS

### Embodiment 1

A method for amplifying a nucleic acid sample, comprising: (a) tagmenting the nucleic acid sample with a population of transposase complexes, wherein the population of transposase complexes comprise: i. a transposase and ii. a set of adaptors of the formula X-Y, wherein: region X is a variable sequence that has a complexity of n, wherein n is at least 3, and region Y is a double-stranded recognition sequence for the transposase, to produce a collection of fragments that are tagged with the variable sequence; and (b) amplifying the tagged fragments using a set of primers, wherein the set of primers comprises at least n different primers and the n different primers collectively hybridize to all of the different sequences of region X, or a complement thereof, to produce amplification products.

### Embodiment 2

The method of embodiment 1, wherein the transposase complexes each comprises a pair of the same adaptor.

### Embodiment 3

The method of embodiment 1, wherein the population of transposase complexes comprises transposase complexes in which the adaptors are different.

### Embodiment 4

The method of any prior embodiment, wherein the nucleic acid sample of step (a) is unamplified genomic DNA.

### Embodiment 5

The method of any prior embodiment, wherein region X is at least partially single stranded.

### Embodiment 6

The method of any prior embodiment, wherein the single stranded part of region X comprises a molecular barcode.

### Embodiment 7

The method of any prior embodiment, further comprising making the ends of the fragments produced in step (a) double stranded prior to amplification Embodiment 8. The method of any prior embodiment, wherein n is in the range of 5 to 40.

### Embodiment 9

The method of any prior embodiment, further comprising sequencing the amplification products of step (b).

### Embodiment 10

The method of any prior embodiment 1, wherein the sequencing is paired end sequencing.

### Embodiment 11

The method of any prior embodiment, wherein the transposase is a Tn5 or Vibhar transposase.

### Embodiment 12

The method of any prior embodiment, wherein the variable sequence is in the range of 6 to 50 nucleotides in length.

### Embodiment 13

The method of any prior embodiment, wherein the produced in step (a) are in the range of 100 bp to 1 kb in length.

### Embodiment 14

The method of any prior embodiment, wherein the amplification is done by PCR.

### Embodiment 15

A kit comprising: (a) a transposase, (b) a set of adaptors of the formula X-Y, wherein: region X is a variable sequence that has a complexity of n, wherein n is at least 3, and region Y is a double-stranded recognition sequence for the transposase, (c) a set of primers, wherein the set of primers comprises at least n different primers and the n different primers collectively hybridize to all of the different sequences of region X, or a complement thereof

### Embodiment 16

The kit of embodiment 15, wherein set of adaptors are present as a mixture in the same container.

### Embodiment 17

The kit of embodiment 15, wherein the adaptors in each set have the same sequence X, and each set of adaptors is present in a different container.

### Embodiment 18

The kit of any prior kit embodiment, wherein the transposase is a Tn5 or Vibhar transposase.

### Embodiment 19

The kit of any prior kit embodiment, wherein region X is at least partially single stranded.

### Embodiment 20

The kit of any prior kit embodiment, wherein the single stranded part of region X comprises a molecular barcode.

### Embodiment 21

The kit of any prior kit embodiment, wherein n is in the range of 5 to 40.

### Embodiment 22

A population of transposase complexes, wherein the population of transposase complexes collectively comprise: i. a transposase; and ii. a set of adaptors of the formula X-Y, wherein: region X is a variable sequence that has a complexity of n, wherein n is at least 3, and region Y is a double-stranded recognition sequence for the transposase.

### Embodiment 23

The population of embodiment 22, wherein the transposase is a Tn5 or Vibhar transposase.

### Embodiment 24

The population of embodiment 22 or 23, wherein region X is at least partially single stranded.

### Embodiment 24

The population of any of embodiments 22-24, wherein the single stranded part of region X comprises a molecular barcode.

### Embodiment 25

The population of any of embodiments 22-25, wherein n is in the range of 5 to 40.

### Example

Aspects of the present teachings can be further understood in light of the following examples, which should not be construed as limiting the scope of the present teachings in any way.

This method has been demonstrated in a tagmentation assay that involved eight distinct primers and their associated indices. These were used to construct a sequencing library of labeled genomic DNA from the equivalent input of 10 human cells, or 60 picograms of DNA.

The library was constructed by the following procedure:
1. The genomic DNA or cells were collected in a buffer with detergent
2. The cell extract was incubated at 50° C. for 10 minutes
3. The extract was exposed to an equimolar mixture of loaded transposases for tagmentation for 20 minutes at 45° C. At the end point, guanodine was added to stop the tagmentation reaction.
4. The DNA library was purified at with SPRI beads.
5. The library was amplified by PCR for 16 cycles.
6. The library as purified again by beads to remove the PCR primers.
7. The library was sequenced on an Illumina MiSeq sequencer using a standard protocol with a paired-end 75-base sequencing kit.

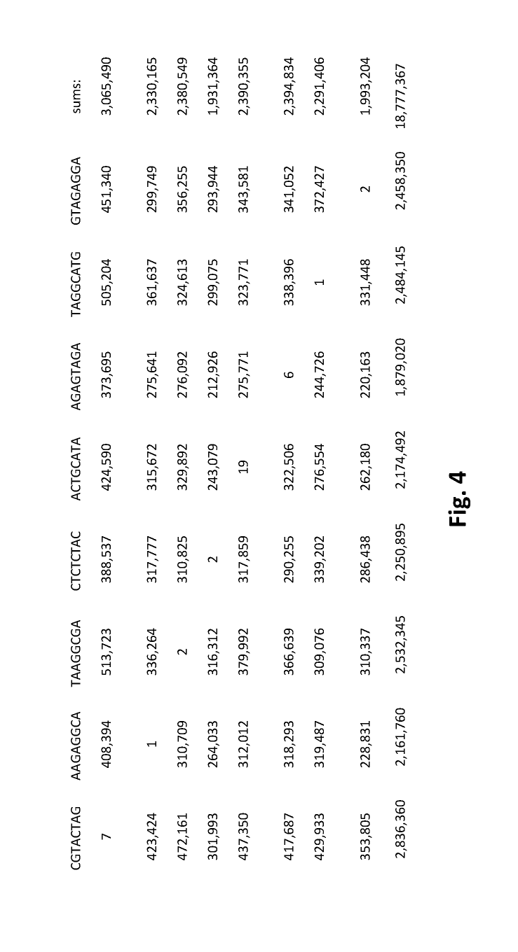

After sequencing, the reads were analyzed to determine the representations of each of the indices and the quality of the genomic information. Out of a total of approximately 26 million read pairs, 19,003,183 aligned to the human genome reference assembly. The majority of these read pairs included index sequences that matched the 8 nominal sequences loaded into transposase enzymes. Those combined representations are given in FIG. 4.

Read pairs with the same index at both ends are not expected because PCR tends to suppress amplification of products with identical ends, as such targets tend to form large stable hairpin loops. Nevertheless, a few were observed, primarily represented in single digits along the diagonal of the table. When the disparities of the representations of each index were examined it was found that overall the highest index (CGTACTAG) is represented about 51-59% more frequently than the lowest. And, when each combination (off-axis term) was examined the highest combination of indices is about 2.3× greater than the lowest. Thus, the indices seem to be represented reasonably uniformly.

Ideally, all primers would be represented at roughly the same level in the sequenced reads. Realistically, there will always be biases that favor some primers while diminishing others. One approach to reduce bias is to test a multitude of distinct primers, and to select those that are the most uniformly represented. Here, 10 primers were tested, and selected 8 of the 10 that were best represented. More optimization could improve the results further. Additionally, more primers can be utilized.

As to performance, if 10,000,000 read pairs are examined, then it was observed that 77% have fragment combined endpoints (cut sites) that are distinct in the set. These 10,000,00 fragments have a mean fragment size of 318 bp and span about 52% of the breadth of the genome.

It will also be recognized by those skilled in the art that, while the invention has been described above in terms of preferred embodiments, it is not limited thereto. Various features and aspects of the above described invention may be used individually or jointly. Further, although the invention has been described in the context of its implementation in a particular environment, and for particular applications those skilled in the art will recognize that its usefulness is not limited thereto and that the present invention can be beneficially utilized in any number of environments and implementations where it is desirable to amplify nucleic acid. Accordingly, the claims set forth below should be construed in view of the full breadth and spirit of the invention as disclosed herein.

1. A method for amplifying a nucleic acid sample, comprising:
(a) tagmenting the nucleic acid sample with a population of transposase complexes, wherein the population of transposase complexes comprises:
i. a transposase and
ii. a set of adaptors of the formula X-Y, wherein:
region X is a variable sequence that has a complexity of n, wherein n is at least 3, and
region Y is a double-stranded recognition sequence for the transposase, to produce a collection of fragments that are tagged with the variable sequence; and
(b) amplifying the tagged fragments using a set of primers, wherein the set of primers comprises at least n different primers and the n different primers collectively hybridize to all of the different sequences of region X, or a complement thereof, to produce amplification products.

2. The method of claim 1, wherein the transposase complexes each comprises a pair of the same adaptor

3. The method of claim 1, wherein the population of transposase complexes comprises transposase complexes in which the adaptors are different.

4. The method of claim 1, wherein the nucleic acid sample of step (a) is unamplified genomic DNA.

5. The method of claim 1, wherein region X is at least partially single stranded.

6. The method of claim 5, wherein the single stranded part of region X comprises a molecular barcode.

7. The method of claim 5, further comprising making the ends of the fragments produced in step (a) double stranded prior to amplification

8. The method of claim 1, wherein n is in the range of 5 to 40.

9. The method of claim 1, further comprising sequencing the amplification products of step (b).

10. The method of claim 1, wherein the sequencing is paired end sequencing.

11. The method of claim 1, wherein the transposase is a Tn5 or Vibhar transposase.

12. The method of claim 1, wherein the variable sequence is in the range of 6 to 50 nucleotides in length.

13. The method of claim 1, wherein the produced in step (a) are in the range of 100 bp to 1 kb in length.

14. The method of claim 1, wherein the amplification is done by PCR.

15. A kit comprising:
(a) a transposase
(b) a set of adaptors of the formula X-Y, wherein:
region X is a variable sequence that has a complexity of n, wherein n is at least 3, and
region Y is a double-stranded recognition sequence for the transposase,
(c) a set of primers, wherein the set of primers comprises at least n different primers and the n different primers collectively hybridize to all of the different sequences of region X, or a complement thereof.

16. The kit of claim 15, wherein set of adaptors are present as a mixture in the same container.

17. The kit of claim 15, wherein the adaptors in each set have the same sequence X, and each set of adaptors is present in a different container.

18. The kit of claim 15, wherein the transposase is a Tn5 or Vibhar transposase.

19. A population of transposase complexes, wherein the population of transposase complexes comprise:
i. a transposase; and
ii. a set of adaptors of the formula X-Y, wherein:
region X is a variable sequence that has a complexity of n, wherein n is at least 3, and
region Y is a double-stranded recognition sequence for the transposase.

20. The population of transposase complexes of claim 19, wherein the transposase is a Tn5 or Vibhar transposase.
